(19) United States
(12) Patent Application Publication
(10) Pub. No.: US 2024/0071613 A1
(43) Pub. Date: Feb. 29, 2024

(54) SYSTEMS AND METHODS FOR AUTOMATIC WORKER HEALTH AND SAFETY ASSESSMENT USING MACHINE LEARNING

(71) Applicant: Honeywell International Inc., Charlotte, NC (US)

(72) Inventors: Justin SCHASSLER, Phoenix, AZ (US); Matthew Damon EMERY, Winchester (GB); Bradley LARSON, Phoenix, AZ (US); Luke ROBERTSON, Peachtree City, GA (US)

(21) Appl. No.: 18/161,538

(22) Filed: Jan. 30, 2023

Related U.S. Application Data
(60) Provisional application No. 63/373,269, filed on Aug. 23, 2022.

(51) Int. Cl. G16H 40/67; A61B 5/00; G06F 21/62; G16H 10/60

(52) U.S. Cl. CPC G16H 40/67; A61B 5/0022; G06F 21/6245; G16H 10/60

(57) ABSTRACT

A safety system for providing a real-time health and safety assessment of a worker performing a task includes a telemetry and video database to store biometric telemetry data and video data of the worker performing the task, an environmental database to store environmental data associated with the worker performing the task, a threshold database to store a threshold for a safety parameter of the worker performing the task, a machine learning-based model to automatically determine the safety parameter of the worker based on the stored biometric telemetry data, video data, environmental data, and threshold, a dashboard to provide access to the stored biometric telemetry data, video data, environmental data, and threshold, and provide the real-time health and safety assessment of the worker based on the determined safety parameter, and a controller to control an operation of the safety system.

### CROSS-REFERENCE TO RELATED APPLICATION(S)

This application claims the benefit of priority to U.S. Provisional Patent Application No. 63/373,269, filed on Aug. 23, 2022, the entirety of which is incorporated by reference herein.

### TECHNICAL FIELD

Various embodiments of the present disclosure relate generally to systems and methods for real time health and safety assessment of a worker, and more particularly, to systems and methods for real time health and safety assessment of a worker using machine learning applied to data aggregated from various sources.

### BACKGROUND

Industrial facilities, such as factories and warehouses, can sometimes involve working conditions that benefit from additional attention to safety. For example, in the aerospace industry, industrial facilities can include maintenance repair and overhaul (MRO) facilities, parts manufacturing, assembly facilities, ground operations, and/or tarmac operations such as baggage handling. Working in industrial settings such as these can involve conditions in which distraction and oversight can potentially create hazards. Situations such as standing too close to machinery, being too poorly hydrated in extreme heat, or poor focus at the wrong time can result in undue risk or safety hazards. In some cases, unpredictable or even uncontrollable factors, such as inclement weather, poor rest, excessive work hours, circadian rhythm issues, or even stress and distraction can contribute to these hazardous situations. Current solutions sometimes involve training of a worker to raise awareness, which can sometimes decrease in effectiveness over time.

The present disclosure is directed to overcoming one or more of these above-referenced challenges.

### SUMMARY OF THE DISCLOSURE

In some aspects, the techniques described herein relate to a safety system for providing a real-time health and safety assessment of a worker performing a task, the safety system including: a telemetry and video database to store biometric telemetry data and video data of the worker performing the task; an environmental database to store environmental data associated with the worker performing the task; a threshold database to store a threshold for a safety parameter of the worker performing the task; a machine learning-based model to automatically determine the safety parameter of the worker based on the stored biometric telemetry data, video data, environmental data, and threshold; a dashboard to provide access to the stored biometric telemetry data, video data, environmental data, and threshold, and provide the real-time health and safety assessment of the worker based on the determined safety parameter; and a controller to control an operation of the safety system.

In some aspects, the techniques described herein relate to a safety system, further including: a schedule database to store schedule information associated with the worker, wherein the machine learning-based model is further configured to automatically determine the safety parameter of the worker based on the stored biometric telemetry data, video data, environmental data, schedule information, and threshold, and wherein the dashboard is further configured to provide access to the stored biometric telemetry data, video data, environmental data, schedule information, and threshold, and provide the real-time health and safety assessment of the worker based on the determined safety parameter.

In some aspects, the techniques described herein relate to a safety system, further including: a data security lock to secure the telemetry and video database, environmental database, threshold database, and dashboard from unauthorized personnel.

In some aspects, the techniques described herein relate to a safety system, wherein the machine learning-based model is trained by: receiving first metadata regarding previous biometric telemetry data, video data, environmental data, and threshold data; extracting a first feature from the received first metadata; receiving second metadata regarding a previous safety incident related to the previous biometric telemetry data, video data, environmental data, and threshold data; extracting a second feature from the received second metadata; and training the machine learning-based model to learn an association between the previous biometric telemetry data, video data, environmental data, and threshold data and the previous safety incident related to the stored biometric telemetry data, video data, environmental data, and threshold data, based on the extracted first feature and the extracted second feature.

In some aspects, the techniques described herein relate to a safety system, wherein the machine learning-based model automatically determines the safety parameter of the worker by extracting a feature from the stored biometric telemetry data, video data, environmental data, and threshold, and by using the extracted feature and a feature of the learned association between the previous biometric telemetry data, video data, environmental data, and threshold data and the previous safety incident related to the previous biometric telemetry data, video data, environmental data, and threshold data.

In some aspects, the techniques described herein relate to a safety system, wherein the real-time health and safety assessment of the worker includes a warning including one or more of the worker is too close to machinery, too poorly hydrated, or has poor focus.

In some aspects, the techniques described herein relate to a safety system, wherein the dashboard further provides a targeted action in response to the real-time health and safety assessment of the worker.

In some aspects, the techniques described herein relate to a method for providing a real-time health and safety assessment of a worker performing a task, the method including: performing, by one or more controllers, operations including: storing biometric telemetry data and video data of the worker performing the task; storing environmental data associated with the worker performing the task; storing a threshold for a safety parameter of the worker performing the task; automatically determining, using a machine learning-based model, the safety parameter of the worker based on the stored biometric telemetry data, video data, environmental data, and threshold; and providing access to the stored biometric telemetry data, video data, environmental data, and threshold, and providing the real-time health and safety assessment of the worker based on the determined safety parameter.

In some aspects, the techniques described herein relate to a method, wherein the operations further include: storing schedule information associated with the worker, wherein the automatically determining the safety parameter of the worker further includes determining, using the machine learning-based model, the safety parameter of the worker based on the stored biometric telemetry data, video data, environmental data, schedule information, and threshold, and wherein the providing access further includes providing access to the stored biometric telemetry data, video data, environmental data, schedule information, and threshold.

In some aspects, the techniques described herein relate to a method, wherein the operations further include: securing the stored biometric telemetry data and video data, stored environmental data, stored threshold, and real-time health and safety assessment from unauthorized personnel.

In some aspects, the techniques described herein relate to a method, wherein the machine learning-based model is trained by: receiving first metadata regarding previous biometric telemetry data, video data, environmental data, and threshold data; extracting a first feature from the received first metadata; receiving second metadata regarding a previous safety incident related to the previous biometric telemetry data, video data, environmental data, and threshold data; extracting a second feature from the received second metadata; and training the machine learning-based model to learn an association between the previous biometric telemetry data, video data, environmental data, and threshold data and the previous safety incident related to the stored biometric telemetry data, video data, environmental data, and threshold data, based on the extracted first feature and the extracted second feature.

In some aspects, the techniques described herein relate to a method, wherein the machine learning-based model automatically determines the safety parameter of the worker by extracting a feature from the stored biometric telemetry data, video data, environmental data, and threshold, and by using the extracted feature and a feature of the learned association between the previous biometric telemetry data, video data, environmental data, and threshold data and the previous safety incident related to the previous biometric telemetry data, video data, environmental data, and threshold data.

In some aspects, the techniques described herein relate to a method, wherein the real-time health and safety assessment of the worker includes a warning including one or more of the worker is too close to machinery, too poorly hydrated, or has poor focus.

In some aspects, the techniques described herein relate to a method, further including: providing a targeted action in response to the real-time health and safety assessment of the worker.

In some aspects, the techniques described herein relate to a non-transitory computer-readable medium storing instructions, that when executed by one or more controllers, perform a method for providing a real-time health and safety assessment of a worker performing a task, the method including: storing biometric telemetry data and video data of the worker performing the task; storing environmental data associated with the worker performing the task; storing a threshold for a safety parameter of the worker performing the task; automatically determining, using a machine learning-based model, the safety parameter of the worker based on the stored biometric telemetry data, video data, environmental data, and threshold; and providing access to the stored biometric telemetry data, video data, environmental data, and threshold, and providing the real-time health and safety assessment of the worker based on the determined safety parameter.

In some aspects, the techniques described herein relate to a non-transitory computer-readable medium, wherein the method further includes: storing schedule information associated with the worker, wherein the automatically determining the safety parameter of the worker further includes determining, using the machine learning-based model, the safety parameter of the worker based on the stored biometric telemetry data, video data, environmental data, schedule information, and threshold, and wherein the providing access further includes providing access to the stored biometric telemetry data, video data, environmental data, schedule information, and threshold.

In some aspects, the techniques described herein relate to a non-transitory computer-readable medium, wherein the method further includes: securing the stored biometric telemetry data and video data, stored environmental data, stored threshold, and real-time health and safety assessment from unauthorized personnel.

In some aspects, the techniques described herein relate to a non-transitory computer-readable medium, wherein the machine learning-based model is trained by: receiving first metadata regarding previous biometric telemetry data, video data, environmental data, and threshold data; extracting a first feature from the received first metadata; receiving second metadata regarding a previous safety incident related to the previous biometric telemetry data, video data, environmental data, and threshold data; extracting a second feature from the received second metadata; and training the machine learning-based model to learn an association between the previous biometric telemetry data, video data, environmental data, and threshold data and the previous safety incident related to the stored biometric telemetry data, video data, environmental data, and threshold data, based on the extracted first feature and the extracted second feature.

In some aspects, the techniques described herein relate to a non-transitory computer-readable medium, wherein the machine learning-based model automatically determines the safety parameter of the worker by extracting a feature from the stored biometric telemetry data, video data, environmental data, and threshold, and by using the extracted feature and a feature of the learned association between the previous biometric telemetry data, video data, environmental data, and threshold data and the previous safety incident related to the previous biometric telemetry data, video data, environmental data, and threshold data.

In some aspects, the techniques described herein relate to a non-transitory computer-readable medium, wherein the real-time health and safety assessment of the worker includes a warning including one or more of the worker is too close to machinery, too poorly hydrated, or has poor focus, and wherein the method further includes providing a targeted action in response to the real-time health and safety assessment of the worker.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary system infrastructure for a safety system for real time health and safety assessment of a worker, according to one or more embodiments.

FIG. 2 depicts an exemplary flow diagram for a safety system for real time health and safety assessment of a worker, according to one or more embodiments.

FIG. 3 depicts an implementation of a computer system that may execute techniques presented herein, according to one or more embodiments.

FIG. 4 depicts a flowchart of a method for providing a real-time health and safety assessment of a worker, according to one or more embodiments.

FIG. 5 depicts a flowchart of a method for providing a real-time health and safety assessment of a worker, according to one or more embodiments.

### DETAILED DESCRIPTION OF EMBODIMENTS

Various embodiments of the present disclosure relate generally to systems and methods for real time health and safety assessment of a worker, and more particularly, to aggregating data from several sources, including wearables and video feeds, to combine with a machine learning model for real time health and safety assessment of a worker performing a task.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section.

As described above, aerospace maintenance repair and overhaul (MRO) facilities, parts manufacturing, ground operations, and/or tarmac operations such as baggage handling, for example, can involve work in settings in which distraction and oversight can create hazards. Situations such as standing too close to machinery, being too poorly hydrated in extreme heat, or poor focus at the wrong time can create hazards. Additionally, human factors such as poor rest, excessive work hours, circadian rhythm issues, or even stress and distraction can sometimes contribute to these hazardous situations in ways that could be recognizable given the right data set. Current solutions often involve training a worker to raise awareness. Although training alone may be cost effective, this solution has the disadvantage of being less effective over time.

This disclosure provides systems and methods to create awareness of these hazardous situations and, ultimately, their mitigation. In particular, this disclosure describes systems and methods for aggregating data from several sources, including wearables and video feeds, to combine with machine learning models for the purpose of real time health and safety assessment of a worker performing a task. The benefits and advantages of the disclosed systems and methods may be in the reduction or prevention of hazards in the workplace. While improved safety is the primary advantage of the disclosed systems and methods, an indirect benefit may also be an increase in worker productivity and performance as managers take targeted actions to reduce stress and external factors which drive the safety assessment.

The disclosed systems and methods may be practiced with a combination of wearable technologies, video feeds, and machine learning software models to assess the conditions of any given worker performing a task. Wearable sensors may be provisioned to collect user biometric telemetry data such as heartrate, temperature, or fatigue, for example, and communicate with a local or cloud hosted safety system and associated with a specific employee. Data may be may be transferred over wired or wireless communication commensurate with the wearable technology, and data may be routed to the safety system. Video feeds may be provisioned for communication with the safety system via secure data interfaces and protocols. Data from video feeds may be captured via common video stream feeds with authenticated and secured channels such that an existing video system can remain intact, and video feed data may be mirrored to the safety system. In the safety system, applications indicate safe, unsafe, and dangerous conditions for a task based on combination of telemetry information from all sources and customer defined thresholds. The machine learning models may be continually updated and trained to be more accurate based on true and false positives that are identified and fed back to the machine learning models. A real-time dashboard may provide data for all active data streams and provide notifications based on an output of the models and algorithms. The real-time dashboard and/or notifications may be displayed for pre-designated supervisory staff on an internal or external user interface.

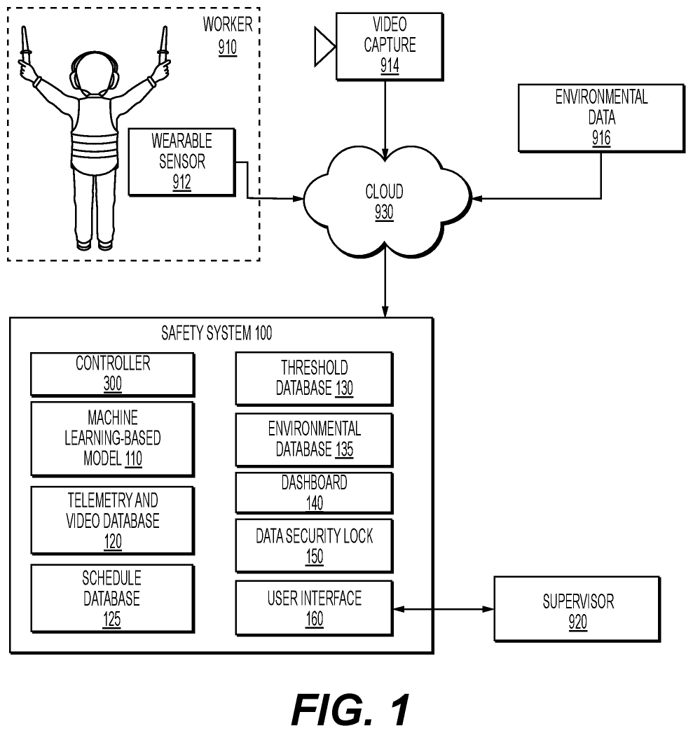

FIG. 1 depicts an exemplary system infrastructure for a safety system for real time health and safety assessment of a worker, according to one or more embodiments. As shown in FIG. 1, safety system 100 may monitor worker 910 using one or more of data provided by wearable sensor 912, video capture system 914, and environmental data system 916, and provide information to one or more of worker 910 or supervisor 920. Wearable sensor 912, video capture system 914, and environmental data system 916 may provide data to safety system 100 through cloud 930. Cloud 930 may be any local or networked system suitable for transferring data.

Wearable sensor 912 may be one or more of a wristband, headband, or electrophysiological monitoring sensor, for example, for collection of biometric telemetry data of worker 910 such as heartrate, temperature, or fatigue, for example. Wearable sensor 912 may periodically or continuously collect user biometric telemetry data, and may send the collected user biometric telemetry data to cloud 930 periodically or continuously. Video capture system 914 may periodically or continuously collect video data of worker 910 performing a task in a surrounding environment, and may send the collected video data to cloud 930 periodically or continuously. Environmental data system 916 may periodically or continuously collect environmental data of worker 910 performing a task in a surrounding environment, and may send the collected environmental data to cloud 930 periodically or continuously. Environmental data may include information such as, for example, a temperature of an environment of worker 910, or a state of a machine that worker 910 is near. However, the disclosure is not limited thereto, and environmental data may include any information that may be relevant to assessing the health and safety of worker 910.

Although safety system 100 is described above as including wearable sensor 912 and video capture system 914, the disclosure is not limited thereto. For example, wearable sensor 912 and video capture system 914 may be provided as a single sensor that is either wearable or remote, or both wearable and remote, or as more than two sensors in any combination of wearable and/or remote. For example, wearable sensor 912 and video capture system 914 may be provided as six wristbands, five pressure pads, and four cameras.

Safety system 100 may include controller 300, machine learning-based model 110, telemetry and video database 120, schedule database 125, threshold database 130, environmental database 135, dashboard 140, data security lock 150, and user interface 160.

Machine learning-based model 110 may use data from one or more of telemetry and video database 120, schedule database 125, threshold database 130, or environmental database 135 to automatically generate a real time health and safety assessment of worker 910. Telemetry and video database 120 may store telemetry and video data associated with worker 910 from wearable sensor 912 and video capture system 914, and may store telemetry and video data associated with other workers from other wearable sensors and video capture systems. Schedule database 125 may store schedule information associated with worker 910, and may store schedule information associated with other workers. Threshold database 130 may store threshold information associated with worker 910, and may store threshold information associated with other workers. The threshold information may include one or more health thresholds and one or more safety thresholds. Environmental database 135 may store environmental information associated with worker 910 from environmental data system 916, and may store environmental information associated with other workers.

Dashboard 140 may provide a software interface for one or more of telemetry and video database 120, schedule database 125, threshold database 130, or environmental database 135. For example, dashboard 140 may receive alerts from machine learning-based model 110 along with real time health and safety assessment of worker 910. Dashboard 140 may receive the real time health and safety assessment of worker 910 from machine learning-based model 110 and generate alerts based on the received assessment.

Data security lock 150 may secure one or more of the telemetry and video database 120, schedule database 125, threshold database 130, environmental database 135, or dashboard 140 from unauthorized personnel. User interface 160 may include a touchscreen display, for example, to provide information to a user from dashboard 140 and receive information from a user to dashboard 140. For example, supervisor 920 may review alerts generated for dashboard 140 and displayed on user interface 160, or may review and/or update worker schedule information in schedule database 125 using user interface 160 to interact with schedule database 125 through dashboard 140.

Safety system 100 may receive data from wearable sensor 912, video capture system 914, and environmental data system 916, and determine a safety condition of worker 910 by analyzing the received data using a safety algorithm, such as a machine learning algorithm trained on one or more of telemetry and video database 120, threshold database 130, or environmental database 135, for example.

For example, telemetry and video database 120 may receive telemetry data from wearable sensor 912. Machine learning-based model 110 may determine the safety condition from the telemetry data that a heartbeat of worker 910 is below a threshold level, as defined in threshold database 130, for worker 910. Consequently, safety system 100 may provide an alert to one or more of worker 910 or supervisor 920 indicating that worker 910 may need immediate medical assistance.

For example, worker 910 may be a pilot of an aircraft, and telemetry and video database 120 may receive telemetry data from wearable sensor 912 worn by the pilot. Machine learning-based model 110 may determine the safety condition from the telemetry data that a heartbeat of the pilot is below a threshold level, as defined in threshold database 130, for the pilot. Consequently, safety system 100 may provide an alert to a cabin crew of the aircraft indicating that the pilot may be asleep or may need immediate medical assistance.

The alert may be one or more of a text message, audio alert, haptic feedback, or visual alert, for example. A visual alert may use different colors such as green, yellow, and red, for example, that correlate respectively with different levels of criticality, such as worker 910 is in a safe condition, worker 910 may be approaching an unsafe condition, and worker 910 needs immediate assistance, for example.

As another example of the safety algorithm, telemetry and video database 120 may receive video data from video capture system 914. Machine learning-based model 110 may determine from the video data that worker 910 is near, as defined in threshold database 130, a machine. Environmental database 135 may receive environmental data from environmental data system 916 providing a state of the machine. Machine learning-based model 110 may determine from the environmental data that the machine that worker 910 is near is currently in use. Telemetry and video database 120 may receive telemetry data from wearable sensor 912 providing information about a heartbeat of worker 910. Machine learning-based model 110 may determine the safety condition from the telemetry data that the heartbeat of worker 910 is below a threshold level, as defined in threshold database 130, for worker 910 near the machine in use. Consequently, safety system 100 may provide an alert to one or more of worker 910 or supervisor 920 to avoid an unsafe interaction of worker 910 with the machine in use.

As another example of the safety algorithm, telemetry and video database 120 may receive video data from video capture system 914. Machine learning-based model 110 may determine from the video data that worker 910 is performing a heat-generating welding operation alone, as defined in threshold database 130 as being a predetermined distance away from or out of view of another worker. Environmental database 135 may receive environmental data from environmental data system 916 providing ambient temperature and humidity of the environment of worker 910. Machine learning-based model 110 may determine from the environmental data that the environment of worker 910 may be hazardous. Telemetry and video database 120 may receive telemetry data from wearable sensor 912 providing information about a hydration state of worker 910. Machine learning-based model 110 may determine the safety condition from the telemetry data that worker 910 is below a threshold hydration state, as defined in threshold database 130, for worker 910 performing a heat-generating task in the hazardous environment while working alone. Consequently, safety system 100 may provide an alert to one or more of worker 910, supervisor 920, or a co-worker to relieve worker 910 in the hazardous situation.

Safety system 100 may receive data from wearable sensor 912 and video capture system 914 and, based on data from schedule database 125, determine a task condition of worker 910 by analyzing the received data using a task algorithm, such as a machine learning algorithm trained on one or more of telemetry and video database 120, schedule database 125, threshold database 130, or environmental database 135, for example. The safety algorithm and task algorithm may be one or more separate algorithms or may be parts of a single algorithm. The safety algorithm and task algorithm may use one or more separate machine learning algorithms or may be parts of a single machine learning algorithm.

As an example of the task algorithm, telemetry and video database 120 may receive video data from video capture system 914. Machine learning-based model 110 may determine from the video data that worker 910 is near, as defined in threshold database 130, a machine. Environmental database 135 may receive environmental data from environmental data system 916 providing a state of the machine. Machine learning-based model 110 may determine from the environmental data that the machine that worker 910 is near is currently in use. Schedule database 125 may contain schedule data that worker 910 has been scheduled to perform a task for three hours, and that a co-worker is currently available to relieve worker 910. Machine learning-based model 110 may determine the task condition from the schedule data that a length of a task for worker 910 has exceeded a threshold level, as defined in threshold database 130, for worker 910 near the machine in use. Consequently, safety system 100 may provide an alert to one or more of worker 910 or supervisor 920 to avoid an unsafe interaction of worker 910 with the machine in use. For example, the alert may provide an alert to supervisor 920 with detailed information including one or more of video data, environmental data, schedule data, and threshold levels. Alternatively or additionally, the alert may include a suggestion that the co-worker is currently available to relieve worker 910. As another example, the alert may limit information provided to personnel, and include only provide an indicator that worker 910 is in a hazardous condition.

The machine learning algorithm that may be useful and effective for the analysis is a neural network, which is a type of supervised machine learning. However, other machine learning techniques and frameworks may be used to perform the methods contemplated by the present disclosure. For example, the systems and methods may be realized using other types of supervised machine learning, such as regression problems or random forest, for example, using unsupervised machine learning such as cluster algorithms or principal component analysis, for example, and/or using reinforcement learning. The algorithm may alternatively or additionally be rule-based.

Supervised machine learning may be useful when safety system 100 is certified, and the machine learning algorithm may be certified and closed so that no further updates are applied. However, the disclosure is not limited thereto, and the machine learning algorithm may be trained in a supervised or unsupervised manner with the databases on a periodic or ongoing basis.

Safety system 100 may use the determined safety condition of worker 910 and the determined task condition of worker 910 to determine a real time health and safety assessment of worker 910 with regard to a currently performed task, and may generate information for one or more of worker 910 or supervisor 920 through dashboard 140 to user interface 160.

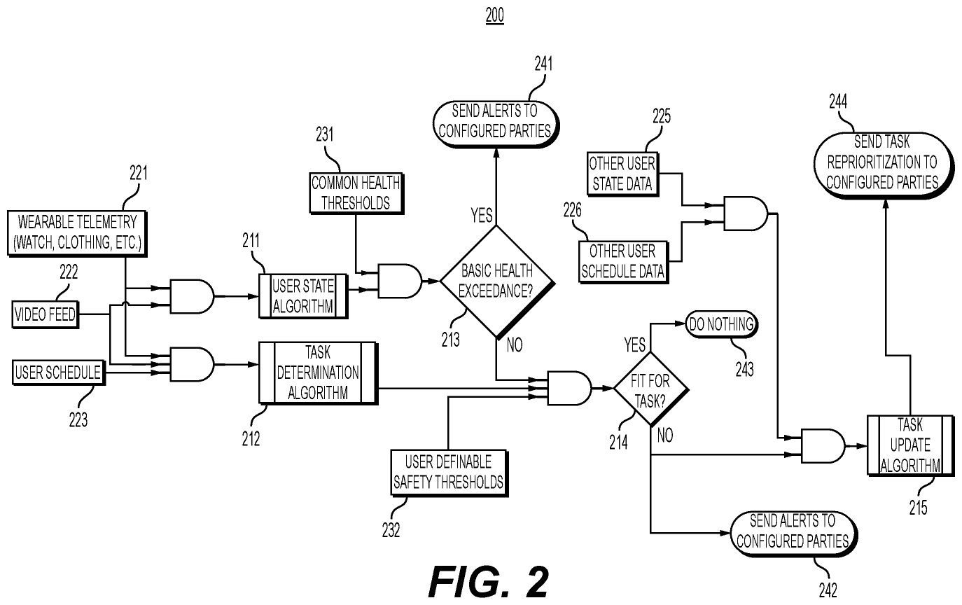

FIG. 2 depicts an exemplary flow diagram for a safety system for real time health and safety assessment of a worker, according to one or more embodiments.

As shown in FIG. 2, flow diagram 200 may illustrate a data flow of safety system 100. In FIG. 2, machine learning-based model 110 may be implemented as user state algorithm 211, task determination algorithm 212, basic health exceedance algorithm 213, task fitness determination algorithm 214, and task update algorithm 215. Each algorithm may include a machine learning-based model or may be components of one or more machine learning-based models. Telemetry and video database 120 and environmental database 135 may be implemented as wearable telemetry 221 and video feed 222. Schedule database 125 may be implemented as user schedule 223, other user state data 225, and other user schedule data 226. Threshold database 130 may be implemented as common health threshold 231 and user safety thresholds 232. Dashboard 140 may be implemented as health alert 241, task alert 242, information output 243, and task re-prioritization 244.

As shown in FIG. 2, data from wearable telemetry 221 and video feed 222 may be input to user state algorithm 211, which may determine a state of worker 910. The state of worker 910 may be compared with common health thresholds 231 in basic health exceedance algorithm 213. For example, telemetry data from wearable telemetry 221 may provide information associated with a heartbeat of worker 910. User state algorithm 211 may determine the state of worker 910 based on the telemetry data from wearable telemetry 221. Basic health exceedance algorithm 213 may determine the safety condition from the state of worker 910 that the heartbeat of worker 910 is below a threshold level, as defined in common health threshold 231, for worker 910. Safety system 100 may then generate health alert 241 to user interface 160. When the comparison in basic health exceedance algorithm 213 indicates a state of worker 910 is within common health threshold 231, safety system 100 may provide the state of worker 910 to task fitness determination algorithm 214.

Additionally, data from wearable telemetry 221, video feed 222, and user schedule 223 may be input to task determination algorithm 212, which may determine a task being performed by worker 910. Task fitness determination algorithm 214 may use the state of worker 910, the task being performed by worker 910, and user safety thresholds 232 to determine whether worker 910 is fit for the task being performed. When task fitness determination algorithm 214 determines worker 910 is fit for the task being performed, information output 243 may provide, to user interface 160, information related to worker 910 as determined by safety system 100 in flow diagram 200. When task fitness determination algorithm 214 determines worker 910 is not fit for the task being performed, safety system 100 may generate task alert 242 to user interface 160 and provide the state of worker 910 and the task being performed by worker 910 to task update algorithm 215.

Task update algorithm 215 may use the state of worker 910, the task being performed by worker 910, other user state data 225, and other user schedule data 226 to determine whether to re-prioritize the task for worker 910, such as postponing the task for worker 910 or assigning the task to another worker, and may provide an alert to user interface 160 for supervisor 920, for example.

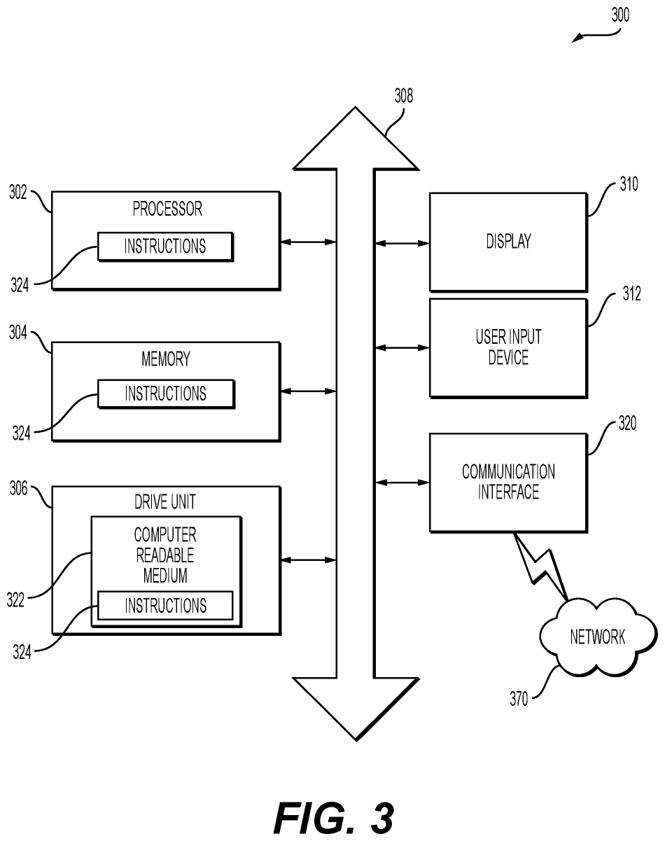

FIG. 3 depicts an implementation of a controller 300 that may execute techniques presented herein, according to one or more embodiments.

The controller 300 may include a set of instructions that can be executed to cause the controller 300 to perform any one or more of the methods or computer based functions disclosed herein. The controller 300 may operate as a standalone device or may be connected, e.g., using a network, to other computer systems or peripheral devices.

In a networked deployment, the controller 300 may operate in the capacity of a server or as a client in a server-client user network environment, or as a peer computer system in a peer-to-peer (or distributed) network environment. The controller 300 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the controller 300 can be implemented using electronic devices that provide voice, video, or data communication. Further, while the controller 300 is illustrated as a single system, the term “system” shall also be taken to include any collection of systems or sub-systems that individually or jointly execute a set, or multiple sets, of instructions to perform one or more computer functions.

As illustrated in FIG. 3, the controller 300 may include a processor 302, e.g., a central processing unit (CPU), a graphics processing unit (GPU), or both. The processor 302 may be a component in a variety of systems. For example, the processor 302 may be part of a standard computer. The processor 302 may be one or more general processors, digital signal processors, application specific integrated circuits, field programmable gate arrays, servers, networks, digital circuits, analog circuits, combinations thereof, or other now known or later developed devices for analyzing and processing data. The processor 302 may implement a software program, such as code generated manually (i.e., programmed).

The controller 300 may include a memory 304 that can communicate via a bus 308. The memory 304 may be a main memory, a static memory, or a dynamic memory. The memory 304 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 304 includes a cache or random-access memory for the processor 302. In alternative implementations, the memory 304 is separate from the processor 302, such as a cache memory of a processor, the system memory, or other memory. The memory 304 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 304 is operable to store instructions executable by the processor 302. The functions, acts or tasks illustrated in the figures or described herein may be performed by the processor 302 executing the instructions stored in the memory 304. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the controller 300 may further include a display 310, such as a liquid crystal display (LCD), an organic light emitting diode (OLED), a flat panel display, a solid-state display, a cathode ray tube (CRT), a projector, a printer or other now known or later developed display device for outputting determined information. The display 310 may act as an interface for the user to see the functioning of the processor 302, or specifically as an interface with the software stored in the memory 304 or in the drive unit 306.

Additionally or alternatively, the controller 300 may include an input device 312 configured to allow a user to interact with any of the components of controller 300. The input device 312 may be a number pad, a keyboard, or a cursor control device, such as a mouse, or a joystick, touch screen display, remote control, or any other device operative to interact with the controller 300.

The controller 300 may also or alternatively include drive unit 306 implemented as a disk or optical drive. The drive unit 306 may include a computer-readable medium 322 in which one or more sets of instructions 324, e.g. software, can be embedded. Further, the instructions 324 may embody one or more of the methods or logic as described herein. The instructions 324 may reside completely or partially within the memory 304 and/or within the processor 302 during execution by the controller 300. The memory 304 and the processor 302 also may include computer-readable media as discussed above.

In some systems, a computer-readable medium 322 includes instructions 324 or receives and executes instructions 324 responsive to a propagated signal so that a device connected to a network 370 can communicate voice, video, audio, images, or any other data over the network 370. Further, the instructions 324 may be transmitted or received over the network 370 via a communication port or interface 320, and/or using a bus 308. The communication port or interface 320 may be a part of the processor 302 or may be a separate component. The communication port or interface 320 may be created in software or may be a physical connection in hardware. The communication port or interface 320 may be configured to connect with a network 370, external media, the display 310, or any other components in controller 300, or combinations thereof. The connection with the network 370 may be a physical connection, such as a wired Ethernet connection or may be established wirelessly as discussed below. Likewise, the additional connections with other components of the controller 300 may be physical connections or may be established wirelessly. The network 370 may alternatively be directly connected to a bus 308.

While the computer-readable medium 322 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 322 may be non-transitory, and may be tangible.

The computer-readable medium 322 can include a solid-state memory such as a memory card or other package that houses one or more non-volatile read-only memories. The computer-readable medium 322 can be a random-access memory or other volatile re-writable memory. Additionally or alternatively, the computer-readable medium 322 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The controller 300 may be connected to a network 370. The network 370 may define one or more networks including wired or wireless networks. The wireless network may be a cellular telephone network, an 802.11, 802.16, 802.20, or WiMAX network. Further, such networks may include a public network, such as the Internet, a private network, such as an intranet, or combinations thereof, and may utilize a variety of networking protocols now available or later developed including, but not limited to TCP/IP based networking protocols. The network 370 may include wide area networks (WAN), such as the Internet, local area networks (LAN), campus area networks, metropolitan area networks, a direct connection such as through a Universal Serial Bus (USB) port, or any other networks that may allow for data communication. The network 370 may be configured to couple one computing device to another computing device to enable communication of data between the devices. The network 370 may generally be enabled to employ any form of machine-readable media for communicating information from one device to another. The network 370 may include communication methods by which information may travel between computing devices. The network 370 may be divided into sub-networks. The sub-networks may allow access to all of the other components connected thereto or the sub-networks may restrict access between the components. The network 370 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure may be implemented using any appropriate techniques for implementing the functionality described herein. The disclosure is not limited to any particular programming language or operating system.

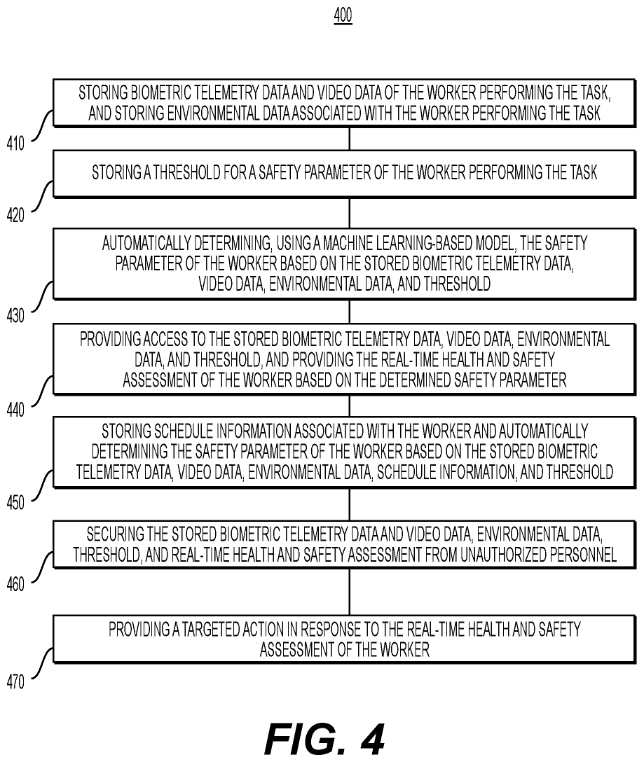

FIG. 4 depicts a flowchart of a method for providing a real-time health and safety assessment of a worker, according to one or more embodiments.

As shown in FIG. 4, method 400 for providing a real-time health and safety assessment of a worker 910 may include performing, by controller 300, various operations. The operations may include storing biometric telemetry data and video data of the worker 910 performing the task, and storing environmental data associated with the worker 910 performing the task (operation 410). Biometric telemetry data and video data may include biometric telemetry data of worker 910 such as heartrate, temperature, or fatigue, for example. Video data of the worker 910 performing the task may be periodically or continuously collected, and may include information associated with video data of worker 910 performing a task in a surrounding environment. The operations may include storing a threshold for a safety parameter of the worker 910 performing the task (operation 420). The threshold may include one or more health thresholds. The threshold may include one or more safety thresholds.

The operations may include automatically determining, using a machine learning-based model 110, the safety parameter of the worker 910 based on the stored biometric telemetry data, video data, environmental data, and threshold (operation 430). For example, telemetry and video database 120 may receive video data from video capture system 914. Machine learning-based model 110 may determine from the video data that worker 910 is near, as defined in threshold database 130, a machine. Environmental database 135 may receive environmental data from environmental data system 916 providing a state of the machine. Machine learning-based model 110 may determine from the environmental data that the machine that worker 910 is near is currently in use. Telemetry and video database 120 may receive telemetry data from wearable sensor 912 providing information about a heartbeat of worker 910. Machine learning-based model 110 may determine the safety parameter from the telemetry data that the heartbeat of worker 910 is below a threshold level, as defined in threshold database 130, for worker 910 near the machine in use.

The operations may include providing access to the stored biometric telemetry data, video data, environmental data, and threshold, and providing the real-time health and safety assessment of the worker 910 based on the determined safety parameter (operation 440). For example, dashboard 140 may provide a software interface for one or more of telemetry and video database 120, schedule database 125, threshold database 130, or environmental database 135. For example, dashboard 140 may receive alerts from machine learning-based model 110 along with real time health and safety assessment of worker 910. Dashboard 140 may receive the real time health and safety assessment of worker 910 from machine learning-based model 110 and generate alerts based on the received assessment.

The operations may include storing schedule information associated with the worker 910, and automatically determining the safety parameter of the worker based on the stored biometric telemetry data, video data, environmental data, schedule information, and threshold (operation 450). The real-time health and safety assessment of the worker 910 may include a warning including one or more of the worker 910 is too close to machinery, too poorly hydrated, or has poor focus. For example, telemetry and video database 120 may receive video data from video capture system 914. Machine learning-based model 110 may determine from the video data that worker 910 is near, as defined in threshold database 130, a machine. Environmental database 135 may receive environmental data from environmental data system 916 providing a state of the machine. Machine learning-based model 110 may determine from the environmental data that the machine that worker 910 is near is currently in use. Schedule database 125 may contain schedule data that worker 910 has been scheduled to perform a task for three hours. Machine learning-based model 110 may determine the safety parameter from the schedule data that a length of a task for worker 910 has exceeded a threshold level, as defined in threshold database 130, for worker 910 near the machine in use.

The operations may include securing the stored biometric telemetry data and video data, stored environmental data, stored threshold, and real-time health and safety assessment from unauthorized personnel (operation 460). The operations may include providing a targeted action in response to the real-time health and safety assessment of the worker 910 (operation 470). For example, safety system 100 may provide an alert to one or more of worker 910 or supervisor 920 to avoid an unsafe interaction of worker 910 with the machine in use. The alert may be one or more of a text message, audio alert, haptic feedback, or visual alert, for example. A visual alert may use different colors such as green, yellow, and red, for example, that correlate respectively with different levels of criticality, such as worker 910 is in a safe condition, worker 910 may be approaching an unsafe condition, and worker 910 needs immediate assistance, for example.

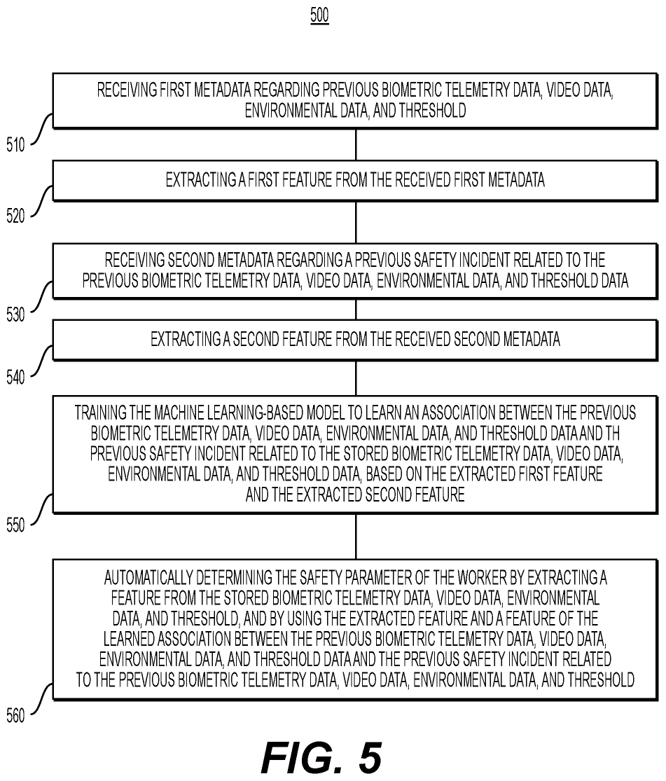

FIG. 5 depicts a flowchart of a method 500 for providing a real-time health and safety assessment of a worker 910, according to one or more embodiments.

The operations of method 400 may also include various operations as illustrated in method 500. In method 500, the machine learning-based model 110 may be trained by receiving first metadata regarding previous biometric telemetry data, video data, environmental data, and threshold data (operation 510), extracting a first feature from the received first metadata (operation 520), receiving second metadata regarding a previous safety incident related to the previous biometric telemetry data, video data, environmental data, and threshold data (operation 530), extracting a second feature from the received second metadata (operation 540), and training the machine learning-based model to learn an association between the previous biometric telemetry data, video data, environmental data, and threshold data and the previous safety incident related to the stored biometric telemetry data, video data, environmental data, and threshold data, based on the extracted first feature and the extracted second feature (operation 550).

The machine learning-based model 110 may automatically determine the safety parameter of the worker 910 by extracting a feature from the stored biometric telemetry data, video data, environmental data, and threshold, and by using the extracted feature and a feature of the learned association between the previous biometric telemetry data, video data, environmental data, and threshold data and the previous safety incident related to the previous biometric telemetry data, video data, environmental data, and threshold data (operation 560).

The disclosure describes systems and methods for aggregating data from several sources, including wearables and video feeds, to combine with machine learning models for the purpose of real time health and safety assessment of a worker performing a task. The benefits and advantages of the disclosed systems and methods may be in the prevention of serious injuries or death of a worker. While safety is the primary advantage of the disclosed systems and methods, an indirect benefit may also be an increase in worker performance as managers take targeted actions to reduce stress and external factors which drive the safety assessment

Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A safety system for providing a real-time health and safety assessment of a worker performing a task, the safety system comprising:
a telemetry and video database to store biometric telemetry data and video data of the worker performing the task;
an environmental database to store environmental data associated with the worker performing the task;
a threshold database to store a threshold for a safety parameter of the worker performing the task;
a machine learning-based model to automatically determine the safety parameter of the worker based on the stored biometric telemetry data, video data, environmental data, and threshold;
a dashboard to provide access to the stored biometric telemetry data, video data, environmental data, and threshold, and provide the real-time health and safety assessment of the worker based on the determined safety parameter; and
a controller to control an operation of the safety system.

2. The safety system of claim 1, further comprising:
a schedule database to store schedule information associated with the worker,
wherein the machine learning-based model is further configured to automatically determine the safety parameter of the worker based on the stored biometric telemetry data, video data, environmental data, schedule information, and threshold, and
wherein the dashboard is further configured to provide access to the stored biometric telemetry data, video data, environmental data, schedule information, and threshold, and provide the real-time health and safety assessment of the worker based on the determined safety parameter.

3. The safety system of claim 1, further comprising:
a data security lock to secure the telemetry and video database, environmental database, threshold database, and dashboard from unauthorized personnel.

4. The safety system of claim 1, wherein the machine learning-based model is trained by:
receiving first metadata regarding previous biometric telemetry data, video data, environmental data, and threshold data;
extracting a first feature from the received first metadata;
receiving second metadata regarding a previous safety incident related to the previous biometric telemetry data, video data, environmental data, and threshold data;
extracting a second feature from the received second metadata; and
training the machine learning-based model to learn an association between the previous biometric telemetry data, video data, environmental data, and threshold data and the previous safety incident related to the stored biometric telemetry data, video data, environmental data, and threshold data, based on the extracted first feature and the extracted second feature.

5. The safety system of claim 4, wherein the machine learning-based model automatically determines the safety parameter of the worker by extracting a feature from the stored biometric telemetry data, video data, environmental data, and threshold, and by using the extracted feature and a feature of the learned association between the previous biometric telemetry data, video data, environmental data, and threshold data and the previous safety incident related to the previous biometric telemetry data, video data, environmental data, and threshold data.

6. The safety system of claim 1, wherein the real-time health and safety assessment of the worker includes a warning including one or more of the worker is too close to machinery, too poorly hydrated, or has poor focus.

7. The safety system of claim 1, wherein the dashboard further provides a targeted action in response to the real-time health and safety assessment of the worker.

8. A method for providing a real-time health and safety assessment of a worker performing a task, the method comprising:
performing, by one or more controllers, operations including:
storing biometric telemetry data and video data of the worker performing the task;
storing environmental data associated with the worker performing the task;
storing a threshold for a safety parameter of the worker performing the task;
automatically determining, using a machine learning-based model, the safety parameter of the worker based on the stored biometric telemetry data, video data, environmental data, and threshold; and
providing access to the stored biometric telemetry data, video data, environmental data, and threshold, and providing the real-time health and safety assessment of the worker based on the determined safety parameter.

9. The method of claim 8, wherein the operations further comprise:
storing schedule information associated with the worker,
wherein the automatically determining the safety parameter of the worker further includes determining, using the machine learning-based model, the safety parameter of the worker based on the stored biometric telemetry data, video data, environmental data, schedule information, and threshold, and
wherein the providing access further includes providing access to the stored biometric telemetry data, video data, environmental data, schedule information, and threshold.

10. The method of claim 8, wherein the operations further comprise:
securing the stored biometric telemetry data and video data, stored environmental data, stored threshold, and real-time health and safety assessment from unauthorized personnel.

11. The method of claim 8, wherein the machine learning-based model is trained by:
receiving first metadata regarding previous biometric telemetry data, video data, environmental data, and threshold data;
extracting a first feature from the received first metadata;
receiving second metadata regarding a previous safety incident related to the previous biometric telemetry data, video data, environmental data, and threshold data;
extracting a second feature from the received second metadata; and
training the machine learning-based model to learn an association between the previous biometric telemetry data, video data, environmental data, and threshold data and the previous safety incident related to the stored biometric telemetry data, video data, environmental data, and threshold data, based on the extracted first feature and the extracted second feature.

12. The method of claim 11, wherein the machine learning-based model automatically determines the safety parameter of the worker by extracting a feature from the stored biometric telemetry data, video data, environmental data, and threshold, and by using the extracted feature and a feature of the learned association between the previous biometric telemetry data, video data, environmental data, and threshold data and the previous safety incident related to the previous biometric telemetry data, video data, environmental data, and threshold data.

13. The method of claim 8, wherein the real-time health and safety assessment of the worker includes a warning including one or more of the worker is too close to machinery, too poorly hydrated, or has poor focus.

14. The method of claim 8, further comprising:
providing a targeted action in response to the real-time health and safety assessment of the worker.

15. A non-transitory computer-readable medium storing instructions, that when executed by one or more controllers, perform a method for providing a real-time health and safety assessment of a worker performing a task, the method comprising:
storing biometric telemetry data and video data of the worker performing the task;
storing environmental data associated with the worker performing the task;
storing a threshold for a safety parameter of the worker performing the task;
automatically determining, using a machine learning-based model, the safety parameter of the worker based on the stored biometric telemetry data, video data, environmental data, and threshold; and
providing access to the stored biometric telemetry data, video data, environmental data, and threshold, and providing the real-time health and safety assessment of the worker based on the determined safety parameter.

16. The non-transitory computer-readable medium of claim 15, wherein the method further comprises:
storing schedule information associated with the worker,
wherein the automatically determining the safety parameter of the worker further includes determining, using the machine learning-based model, the safety parameter of the worker based on the stored biometric telemetry data, video data, environmental data, schedule information, and threshold, and
wherein the providing access further includes providing access to the stored biometric telemetry data, video data, environmental data, schedule information, and threshold.

17. The non-transitory computer-readable medium of claim 15, wherein the method further comprises:
securing the stored biometric telemetry data and video data, stored environmental data, stored threshold, and real-time health and safety assessment from unauthorized personnel.

18. The non-transitory computer-readable medium of claim 15, wherein the machine learning-based model is trained by:
receiving first metadata regarding previous biometric telemetry data, video data, environmental data, and threshold data;
extracting a first feature from the received first metadata;
receiving second metadata regarding a previous safety incident related to the previous biometric telemetry data, video data, environmental data, and threshold data;
extracting a second feature from the received second metadata; and
training the machine learning-based model to learn an association between the previous biometric telemetry data, video data, environmental data, and threshold data and the previous safety incident related to the stored biometric telemetry data, video data, environmental data, and threshold data, based on the extracted first feature and the extracted second feature.

19. The non-transitory computer-readable medium of claim 18, wherein the machine learning-based model automatically determines the safety parameter of the worker by extracting a feature from the stored biometric telemetry data, video data, environmental data, and threshold, and by using the extracted feature and a feature of the learned association between the previous biometric telemetry data, video data, environmental data, and threshold data and the previous safety incident related to the previous biometric telemetry data, video data, environmental data, and threshold data.

20. The non-transitory computer-readable medium of claim 15,
wherein the real-time health and safety assessment of the worker includes a warning including one or more of the worker is too close to machinery, too poorly hydrated, or has poor focus, and
wherein the method further includes providing a targeted action in response to the real-time health and safety assessment of the worker.
